(19) United States
(12) Patent Application Publication
(10) Pub. No.: US 2018/0339096 A1
(43) Pub. Date: Nov. 29, 2018

(54) ERGONOMIC GIRDLE FOR PERSONS ON PERITONEAL DIALYSIS

(71) Applicant: NUTRICION NIN S.A. DE C.V., Guadalajara, Jalisco (MX)

(72) Inventors: Luis Gerardo Gonzalez Delgado, Guadalajara, Jalisco (MX); Fabiola Martin Del Campo Lopez, Guadalajara, Jalisco (MX); Cristina Morgado Escalante, Guadalajara, Jalisco (MX)

(21) Appl. No.: 15/777,904

(22) PCT Filed: Nov. 16, 2016
(86) PCT No.: PCT/MX2016/000117

§ 371 Date: May 21, 2018

(30) Foreign Application Priority Data
Nov. 20, 2015 (MX) MX/U/2015/000568

(51) Int. Cl. A61M 1/28

(52) U.S. Cl. CPC A61M 1/285; A61M 2209/088

(57) ABSTRACT

The mentioned invention describes an ergonomic belt for persons with peritoneal dialysis which is used for care of the patients after having surgery, specifically for kidney failure, where a catheter is placed to carry out the dialysis, the benefits of the usage of the mentioned invention provide abdominal support allowing to ensure the care of the suture, so it won't open because of the movement or the patient's efforts.

It also has the particularity to protect the catheter, since there is an orifice that could be outside of the belt preventing for it to bend or to be pulled under the weight of the dialysis fluids, also having a compartment where the catheter could be located protecting at the same time from the environment exposition and the perspiration of the patient, so it can be assured not having infections that could harm the integrity of the person carrying it.

### TECHNICAL FIELD OF INVENTION

The following invention is related with the technical field of the medical devices and the technological medical equipments since it contributes an ergonomic belt for persons with peritoneal dialysis.

### BACKGROUND OF THE INVENTION

There are several products on the market that are used to give support, to assure that a surgical suture is not exposed to open or to get infected and at the same time it helps to reduce discomfort on the patients that had been submitted to an abdominal surgery, specifically when they are treated for kidney failure, where, the postsurgical method is the placement of a catheter, which helps the introduction of a saline solution through the catheter and later its output once the toxins exchange is finished.

The peritoneal dialysis is a procedure that allows to debug liquids and electrolytes on patients that are treated from kidney failure; the peritoneal dialysis use a natural membrane (the peritoneum) as a filter. The dialysis fluid is introduced into the peritoneal cavity through a flexible tube that previously was introduced on the abdomen in a permanent way during a minor surgical intervention; part of this tube or catheter stayed out of the abdomen; thus, allows to connect it to the saline solution bags. The catheter is hidden under clothing.

The installed catheter it is known as the dialysis catheter, but name is Tenckhoff catheter in honor of the doctor that described this system of dialysis.

The peritoneal dialysis eliminates organic substances, metabolism products, when the kidneys do not function appropriately, eliminating toxic substances from the organism, like excess endogenous liquids, which would be excreted by the kidneys if it were functioning properly, it helps to correct. electrolyte imbalances and to reduce the edema.

As long as the liquid is on the peritoneal cavity, dialysis occurs, the excess of liquids and the waste products pass from the blood, through the peritoneal membrane, to the dialysis fluid.

The solution is changed periodically, and this process is called “exchange”. There are two peritoneal dialysis modalities (Continuous Ambulatory Peritoneal Dialysis and Automated Peritoneal Dialysis).

When performing the peritoneal dialysis, in any of its modalities,it is important to do the exchanges on a clean a d air stream free area, since there is a risk of infection.

The Peritonitis is the most common serious complication. The infections on the exit area of the catheter or “the tunnel” (route of the peritoneum to the exit area) are less serious, but more frequent.

There are three types of peritoneal dialysis, which are:

i. The continuous ambulatory peritoneal dialysis, it is called continuous because there are always fluids in the peritoneum and ambulatory because the patient does it at home; it is performed manually and after finishing the exchange the bag system is discarded. Most of the patients on this modality need to do 3 or 4 daily exchanges; the fluid drainage requires 10 to 20 minutes and the infusion of the new solution between 5 to 10 minutes; the learning requires between one and two weeks and does not require needles. The treatment can be performed without leaving the house, it is flexible and can be adjusted to different needs and schedules and it is possible to choose the moment to perform the exchanges.

ii. The automated peritoneal dialysis is performed at home, during the night, while sleeping; a device controls the time to perform the necessary exchanges, drain the used solution and introduce the new dialysis solution on the peritoneal cavity. When it is time to sleep, just have to turn the device on and to plug the catheter to the line system, the device will perform the exchanges during 8 to 9 hours, while sleeping; through the morning, the patient would only have had to disconnect from the device. The automated peritoneal dialysis devices are safe, easy to handle and can be used any place with electricity, it is an ideal option for working people, children of school age and to people who need help during to dialysis.

iii. The intermittent peritoneal dialysis is performed on the hospital area, on newly diagnosed patients and that requires an urgent dialysis, in the first instance a rigid catheter is placed, and 30 baths are performed, leaving them in cavity during 30 minutes each one. Also, it is done to patients who doesn't have the properly conditions to perform a continuous ambulatory peritoneal dialysis Each one of this peritoneal dialysis uses the Tenckhoff catheter.

The peritoneal dialysis requires access to the peritoneum, because this access breaks down the normal barriers of the skin, and since the people with kidney failure regularly has a mildly depressed immune system, the infections are relatively common. The fluid of the peritoneal dialysis contains glucose, so if the person in charge do not take care of the cleaning technique, can favor the growth of germs; the frequency of infection of peritoneal fluid on the units of peritoneal dialysis that are experienced is one infection per patient every two or three years; the infections can be located, on the exit area of the catheter or in the tunnel zone under the skin, where the infection is limited to the skin or the soft tissue surrounding the catheter; or can be potentially more severe if the infection reaches the peritoneum. On this case, it is called post-dialysis peritonitis, which may require antibiotics and support care (usually do not require hospital admission) or, if the peritonitis is severe, removal or replacement of the catheter and a change on the modality of renal replacement therapy to hemodialysis. Very exceptionally, the severe peritonitis could be a threat to life; the long term peritoneal dialysis could induce changes on the peritoneal membrane, causing it to do not act more as a dialysis membrane as well as before.

This function loss could manifest as a loss of capacity for the dialysis, or a poorer fluid Exchange (also known as ultrafiltration failure); the loss of function of the peritoneal membrane could advice changes on the technique as one step from the continuous ambulatory peritoneal dialysis to automated techniques such as automated peritoneal dialysis or even to hemodialysis.

Another complications that may occur are fluid leaks on the surrounding soft tissue, often the scrotum on men. The hernias are another problem that may occur due to the abdominal fluid loading.

This often requires repair before the peritoneal dialysis is recommended.

The catheter for peritoneal dialysis is a small silicone tube that is introduced intro the peritoneal cavity through a surgery, the place where the catheter exits is called exit area; it is normal that soon after being installed, exists a slight bleeding or a transparent fluid on the exit area, and it should disappear between one or two weeks as it heals.

So that there are no long term infectious complications, an early post-operative care is required, to avoid the growth of bacteria on the surgical wound and on the exit area.

These cares include:
Daily bath, without getting wet the exit area, of the surgical wound.
Immobilize the catheter, to avoid pulling or twist; because this favors for the exit area to be hurt and to get infected later.
To keep dry the exit area and surgical wound, mustn't get wet at least 10 days after placement.
To change the dressings daily, this must be performed by specialized staff or trained family member using gloves, face mask and sterile technique.
Do not remove or lift the scabs surrounding the exit area.
Do not use chemical substances to clean the catheter of the exit area, such as alcohol, chlorinated substances, isodine or ointments that may damage the catheter and to cause an infection on the peritoneum.
Must not take a bath on tub, or to use jacuzzis, there are too many germs that may cause infections.
Must not place the belt on the exit area.
Do not use scissor near the catheter.

Considering that any of the relevant cares of the renal insufficiency patients, to whom it is necessary to perform dialysis, and to whom a catheter post surgical may be placed, where is recommended the use of support for this item, with the goal of minimize the infection risk or inappropriate conditions because of moving the catheter.

A patent technical search was made, and it was found that a variety of devices has been designed for this purpose, as mentioned on the patent application document US2009192432 A1, published on Jul. 30, 2009, which referred to an improved abdominal belt, which can include at least one external fixing mechanism to hold the drain container. In an embodiment, the fixing mechanism may include one or more types of grips or tie strips. On another embodiment, it is provided an improved abdominal belt that could hold multiple external drain containers. The improved abdominal belt could include besides one or more openings to hold a drain tube from a container fastened to the abdominal belt.

On another hand, the utility model document CN2803393 (Y), published on Aug. 9, 2006, describes an abdominal support strap that is used to protect the abdominal area when the peritoneal dialysis is performed on the patient with chronic renal failure.

The utility model is a cloth belt with a splinter shape with a locking device, the main section of the cloth belt is an elastic part from each side which is connected respectively to a fixed part and the part to protect, a penetration opening of a guide tube is arranged on the protection part and a guide tube bag is sewed on the position of the penetration opening from a guide tube.

The utility model considers an opening to protect the catheter for the peritoneal dialysis, the fixation of the catheter is to avoid it to move, avoiding damages to the catheter for peritoneal dialysis, to improve the situation of the patient that holds the catheter, supporting a pendulum abdominal Wall with the purpose to reduce the pain sensation from the patient, improving the movement capacity, increasing the contact area between the liquid that is introduced to the abdominal area and the peritoneum, improving the effects of the peritoneal dialysis, protecting the waist, warming the kidney, etc.

By last, it refers to the patent application document US3902503 (A), published on Sep. 2, 1975, that describes the surgical belt from a predetermined length to surround and support an element of the body within a wide range of sizes. The belt is characterized for having at least one disposable part to adjust to its length to adapt to a small body size within the range without having an excessive overlap. The belt comprises an elongated base panel with enough length to surround a certain type of body and at least an end panel. Means are provided to ensure on free form the end panel to the base panel to allow the end panel to serve as an extension to accommodate to a larger body size. One first grip is achieved from the base panel, and one second grip is made from each part of the base panel and the end panel in an independent way and freely interconnecting it to the first fixation part.

The end panel can be removed from the belt to reduce the superimposing of it when the user body size does not require the presence of the end panel and the first grip element is interconnected with the second fixation part of the base panel.

The previously mentioned documents do not mention that perspiration of the skin is allowed and that it mold to the body, as well as cannot be configured in such a way that it is resistant, so it can be washed with any product and to helps to avoid problems for the patient sweat to be in contact with the catheter.

All the documents mention of an opening that go through completely the belt, which increases the risk for dust to get in or any polluting substance that could cause an infection in the wound.

### OBJECT OF THE INVENTION

The present invention's purpose is to, provide an ergonomic belt for persons with peritoneal dialysis, where, it is reduced the movement risk unappropriated for the catheter, the possible contamination and infection of the catheter for exposition to the environment or the patient sweat and at the same time to offer a belt that allows the patient comfort after the surgery helping with the recovery of the patient and to reduce the pain because of the adjusted support of such belt.

### BRIEF DESCRIPTION OF THE FIGURES

The characteristic details of this novelty ergonomic belt for persons with peritoneal dialysis are shown clearly on the next description and the accompanying figures, as long as an illustration of it, and following the same reference signs to indicate the shown parts.

However, such figures are shown as an example and should not be considered as limiting for the invention.

FIG. 1 shows a rear view of the ergonomic belt for persons with peritoneal dialysis

FIG. 2 shows a front view of the ergonomic belt for persons with peritoneal dialysis.

FIG. 3 shows a rear perspective view of the ergonomic belt for persons with peritoneal dialysis.

FIG. 4 shows a rear view of a variant for the ergonomic belt for persons with peritoneal dialysis.

FIG. 5 shows a rear view on perspective of a variant for the ergonomic belt for persons with peritoneal dialysis.

FIG. 6 shows a view of the cross section performed on the variant of the ergonomic belt for persons with peritoneal dialysis.

FIG. 7 shows a view on perspective rear of the cross section performed on the variant of the ergonomic belt for persons with peritoneal dialysis.

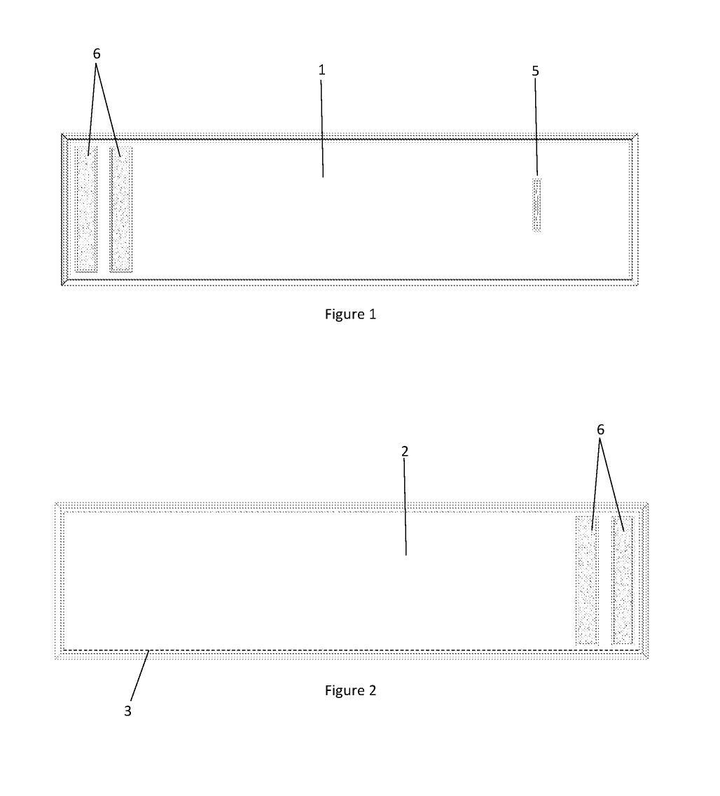

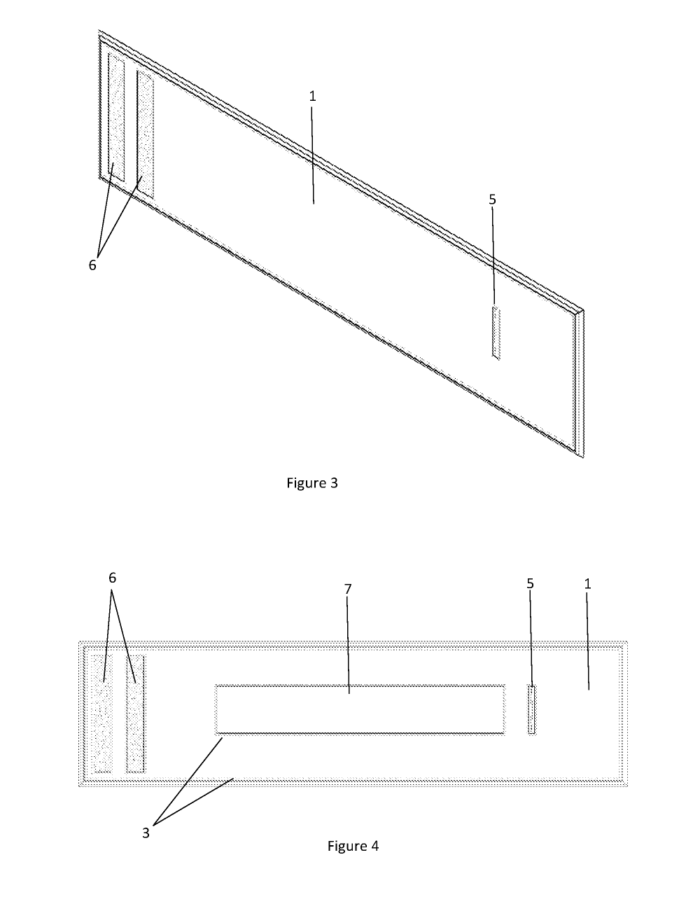

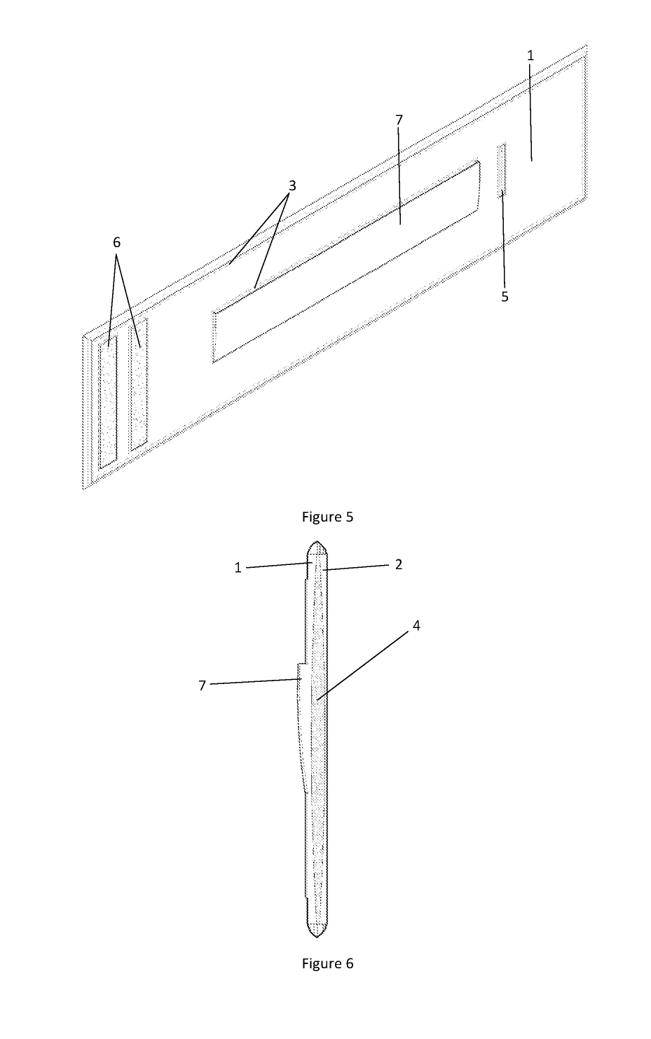

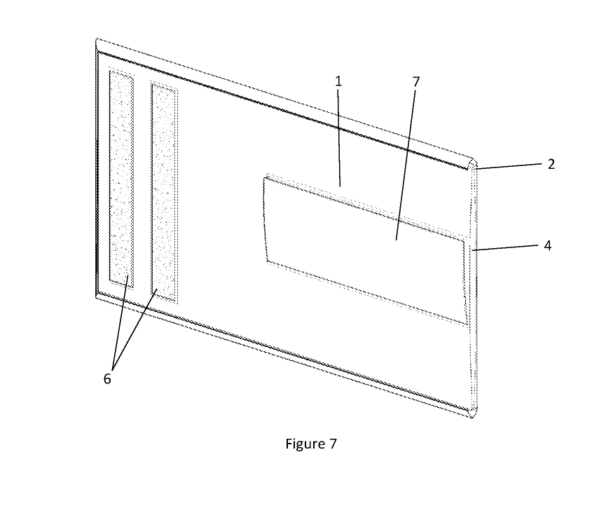

### DETAILED DESCRIPTION OF THE INVENTION

For a better understanding of the invention, below is listed the parts that form the ergonomic belt for persons with peritoneal dialysis.
1. Rear view
2. Front view
3. Reinforcement seam
4. Intermediate space
5. Orifice
6. Clamping mechanism
7. Compartment

The ergonomic belt for persons with peritoneal dialysis, comprises:

A front view (2) configured to adjust and to mold to the patient body while allows the transpiration, such front view (2) is preferably of stretching cloth, to mold and adjust to the user of the belt, which give more security and can be used under the clothes without noticing it, allowing the transpiration of the skin.

A rear view (1) joins the front view (2) through a reinforcement stitching (3) located on the perimeter of both views, which allows to generate between the front view (2) and the rear view (2), an intermediate space (4), which thanks to the orifice (5) that go through the rear view (1), preferably on an end, serves to host a catheter avoiding the exposition with the environment and the body of the patient.

The reinforcement stitching (3) can be of cotton thread, synthetic nylon or a combination of both.

The rear view (1) is always in contact with the patient skin and is fabricated preferably of cotton since is lighter, fresh, hypoallergenic and nice to the human touch, allowing the perspiration skin from the patient, without matter being on elevated temperatures, in case there is sweat from the patient, the fabric allows to absorb it to avoid it to affect the catheter.

A clamping mechanism (6) is located on an end of the front view (2) and another on the rear view (1), on the opposite end where the first one was located; the clamping mechanism (6) could be extended from the end to the middle of any of the views, which allows for the patient with peritoneal dialysis to tie down and fix with the necessary pressure the ergonomic belt for persons with peritoneal dialysis; such capturing mechanism (6) could be velcro, snaps, straps or a combination of the before mentioned.

One of the variables of the mentioned invention are illustrated on the 4,5,6 and 7 figures where can be seen that the rear view (1) has a compartment (7) hold thanks to the stitching reinforcement (3), such compartment (7) it is preferably under the orifice (5), which allows to place the catheter, a drainage pump or whatever is necessary to the patient's recovery.

### Preferential Realization of the Invention

### EXAMPLES

The following examples illustrate a preferably way, to carry out the realization of the mentioned invention, so it shouldn't be considered as limiting of it.

### Example 1

### Abdominal Support and Catheter Hold Located Post Surgery through the Ergonomic Belt for Persons with Peritoneal Dialysis

With the reference of the before mentioned figures, the ergonomic belt for persons with peritoneal dialysis, from the present invention, should be located around the abdominal area of the patient, where initially should be placed the rear view (1) so that could be in contact with the skin and the front view (2) on the opposite site, leaving it exposed to the environment; the catheter goes through the orifice (5) and is located on the compartment (7) or in the middle space (4) which is formed by the union of the front view (2) and the rear view (1), such views are jointed by the reinforcement stitching (3) once it is done, the belt must be fixated to the patient by the clamping system (6) so that the patient is comfortable and the catheter has no movement.

1. An ergonomic belt for persons with peritoneal dialysis, comprising:
a front portion configured to fit and mold to a body;
a rear portion joined to the front portion through a reinforcement stitching;
a space between the front portion and the rear portion;
an orifice that goes through the rear portion; and,
a clamping mechanism located on the front portion and the rear portion.

2. The belt of claim 1, wherein the front portion comprises a stretching cloth.

3. The belt of claim 1 wherein the reinforcement stitching is located on the perimeter of the front portion and the rear portion.

4. The belt of claim 3, wherein the reinforcement stitching comprises thread.

5. The belt of claim 4 where the thread is a material selected from the group consisting of cotton, synthetic nylon and a combination of both cotton and synthetic nylon.

6. The belt of claim 1 wherein the rear portion comprises cotton.

7. The belt of claim 1 wherein the clamping system is located on an end of the front portion and the opposite end of the rear portion.

8. The belt of claim 7, wherein the clamping mechanism extends from an end to a middle of any of the portions.

9. The belt of claim 7, wherein the clamping mechanism comprises a fastener selected from the group consisting of velcro, stamps, straps and a combination of velcro, stamps and straps.

10. The belt of claim 9, wherein the rear portion has a compartment.

11. The belt of claim 10, wherein the compartment is attached to a reinforcement stitching.

12. The belt of claim 10, wherein the compartment is located preferably under the orifice.
